(19) United States
(12) Patent Application Publication
(10) Pub. No.: US 2004/0054760 A1
(43) Pub. Date: Mar. 18, 2004

(54) Deployable telemedicine system

(76) Inventors: Richard E. Ewing, College Station, TX (US); James H. Duke, Houston, TX (US); James A. Wall, Bryan, TX (US); Larry D. Flournoy, Houston, TX (US); Christopher J. Kocmoud, College Station, TX (US); Zhihua Xu, College Station, TX (US); Jose?apos; Salinas, College Station, TX (US); Robert D. Tindall, Pearland, TX (US)

Correspondence Address: BAKER BOTTS L.L.P., 2001 ROSS AVENUE, SUITE 600, DALLAS, TX, 75201-2980, US

(21) Appl. No.: 10/449,254

(22) Filed: May 30, 2003

Related U.S. Application Data
(60) Provisional application No. 60/385,245, filed on May 31, 2002.
(60) Provisional application No. 60/385,260, filed on May 31, 2002.

(51) Int. Cl. G06F 15/16; G06K 9/00

(52) U.S. Cl. 709/219; 382/128

(57) ABSTRACT

A telemedicine system for communicating medical information includes portable modules that receive medical information associated with one or more patients. The portable modules have devices including a medical device that collects the medical information comprising medical data, an audio-video device that collects the medical information comprising multimedia data, and a communication device that communicates the medical information to a station in real time.

### RELATED APPLICATIONS

This application claims benefit under 35 U.S.C. § 119(e) of U.S. Provisional Application Serial No. 60/385,260, entitled “THE DEPLOYABLE TELEMEDICINE SYSTEM SPECIFICATIONS,” Attorney's Docket 017575.0759, filed May 31, 2002, and of U.S. Provisional Application Serial No. 60/385,245, entitled “COMMUNICATION MANAGER SPECIFICATIONS,” Attorney's Docket 017575.0760, filed May 31, 2002.

### GOVERNMENT FUNDING

The U.S. Government may have certain rights in this invention as provided for by the terms of Grant Nos. DAMD 17-00-2-0010, DAMD 17-98-2-8002, and DAMD 17-01-2-0054 awarded by U.S. Army Medical Research and Material Command at Fort Detrick, Md.

### TECHNICAL FIELD OF THE INVENTION

This invention relates generally to the field of telemedicine and more specifically to a deployable telemedicine system.

### BACKGROUND OF THE INVENTION

Providing medical care to a patient at a remote location may involve gathering and communicating medical information about the patient. Known techniques for gathering and communicating medical information, however, are typically inefficient and not comprehensive. Accordingly, known techniques for gathering and communicating medical information may be unsatisfactory in certain situations.

### SUMMARY OF THE INVENTION

In accordance with the present invention, disadvantages and problems associated with previous techniques for gathering and communicating medical information may be reduced or eliminated.

According to one embodiment of the present invention, a telemedicine system for communicating medical information includes portable modules that receive medical information associated with one or more patients. The portable modules have devices including a medical device that collects the medical information comprising medical data, an audio-video device that collects the medical information comprising multimedia data, and a communication device that communicates the medical information to a station in real time. According to another embodiment, the portable modules have devices that include a power supply system that provides current and voltage from AC sources, DC sources, or both.

Certain embodiments of the invention may provide one or more technical advantages. A technical advantage of one embodiment may be that medical information may be communicated using heterogeneous communication links. A technical advantage of another embodiment may be that the medical information may be gathered using a portable system. A technical advantage of yet another embodiment may be that medical and multimedia data may be communicated in real time.

Certain embodiments of the invention may include none, some, or all of the above technical advantages. One or more other technical advantages may be readily apparent to one skilled in the art from the figures, descriptions, and claims included herein.

### BRIEF DESCRIPTION OF THE DRAWINGS

For a more complete understanding of the present invention and its features and advantages, reference is now made to the following description, taken in conjunction with the accompanying drawings, in which:

FIG. 1 is a block diagram of one embodiment of a system for communicating information;

FIG. 2 is a block diagram illustrating one embodiment of a communications manager that may be used with the system of FIG. 1;

FIG. 3 is a flowchart illustrating one embodiment of a method for communicating data packets that may be used by the communications manager of FIG. 2;

FIG. 4 is a block diagram illustrating one embodiment of a paramedic station and a physician station that may be used with the system of FIG. 1;

FIG. 5 is a diagram illustrating one embodiment of a graphical user interface (GUI) that may be used at the paramedic station of FIG. 4;

FIG. 6 is a diagram illustrating one embodiment of a, graphical user interface (GUI) that may be used at physician station 24 of FIG. 5; and

FIG. 7 illustrates one embodiment of a telemedicine system that may be used to gather and communicate medical information.

### DETAILED DESCRIPTION OF THE DRAWINGS

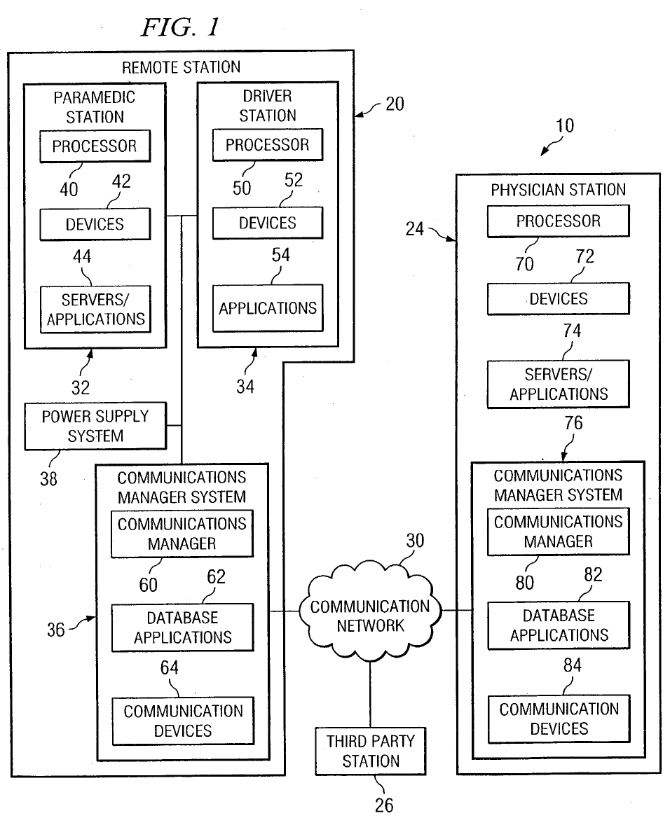

Embodiments of the present invention and its advantages are best understood by referring to FIGS. 1 through 7 of the drawings, like numerals being used for like and corresponding parts of the various drawings.

FIG. 1 is a block diagram of one embodiment of a system 10 for communicating information. System 10 may communicate medical information about a patient from a remote station, which may be located, for example, on a battlefield or at an emergency scene. System 10 may also be used to receive instructions from a physician located a distance away from the remote station. System 10 may use communication links such as wireless and/or terrestrial links to provide real time communication of medical information comprising multimedia and other data. According to one embodiment, system 10 may include a communications manager that is operable to multiplex heterogeneous communication links. According to another embodiment, the remote station may be located in a deployable telemedicine system.

According to the illustrated embodiment, system 10 includes a remote station 20, a physician station 24, a third-party station 26, and a communication network 30 coupled as shown in FIG. 1. Remote station 20 may be used to provide medical and other information about a patient, and may be located anywhere a patient may be located, for example, an ambulance, a battlefield, or a clinic, and may provide medical information about the patient to a physician located at physician station 24. Third-party station 26 may provide additional medical information such as the medical history of the patient to remote station 20, physician station 24, or both.

According to the illustrated embodiment, remote station 20 includes a paramedic station 32, a driver station 34, a communications manager system 36, and a power supply system 38 coupled as shown in FIG. 1. Paramedic station 32 may be used to collect and communicate medical information about the patient. According to the illustrated embodiment, a paramedic may operate paramedic station 32 to collect the information. Any suitable user, however, may operate paramedic station 32 without departing from the scope of the invention. The patient may comprise any living entity for which medical treatment may be provided, for example, a human. Medical information may include any medical data describing the physical or mental condition of the patient, the diagnosis or treatment of the patient, or both, for example, audio, video, or textual data about the current or past condition of the patient. Medical information may also include information used to obtain the medical data, such as a patient identifier.

According to the illustrated embodiment, paramedic station 32 includes a processor 40, devices 42, and servers/applications 44. Processor 40 controls the operation of paramedic station 32, and may comprise any suitable device operable to accept input, process the input according to predefined rules, and produce output, for example, a personal computer, workstation, network computer, wireless data port, wireless telephone, personal digital assistant, central processing unit, or any other suitable processing device.

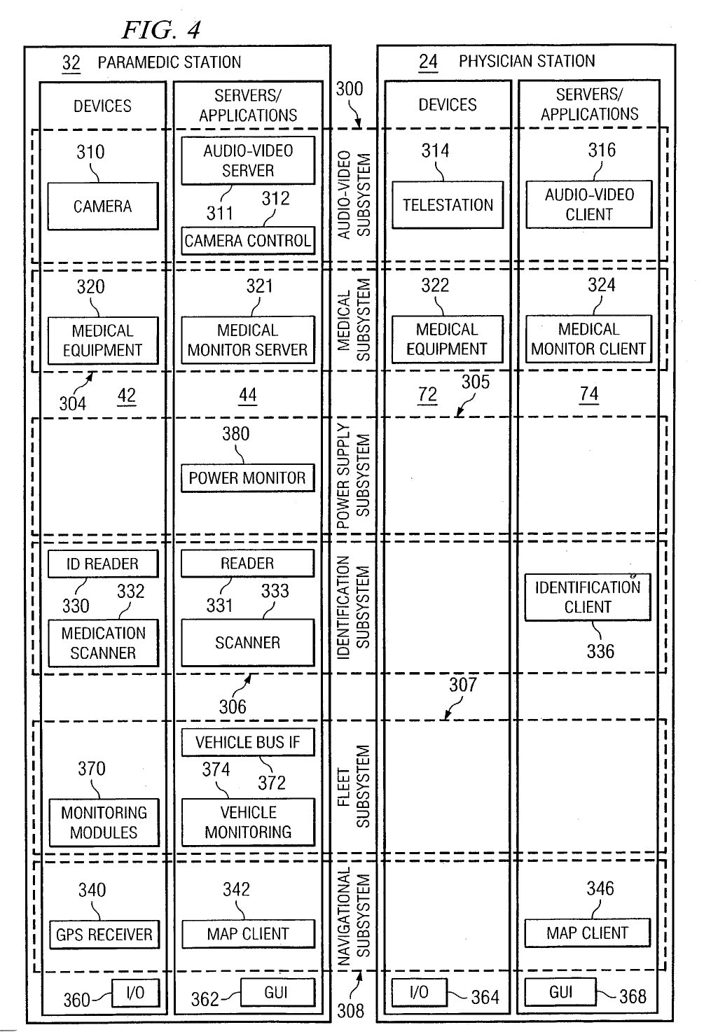

Devices 42 may include medical equipment or other devices operable to collect and communicate information about the patient. Servers/applications 44 may include logic such as hardware or software that may be used by processor 40 or devices 42 to collect and communicate the information. Servers/applications 44 and 74 may include applications developed using the JAVA programming language, any of the C family programming languages, or other suitable programming language. Any suitable operating system such as Microsoft Windows and/or Linux may be used for system 10. An embodiment of paramedic station 32 is described in more detail with reference to FIG. 4.

Driver station 34 may be used to communicate information to and from a driver of a vehicle, such as an ambulance. According to the illustrated embodiment, driver station 34 includes a processor 50, devices 52, and applications 54. Devices 52 may include, for example, an input-output device such as a computerized tablet that the driver may use to record information about the run made by the ambulance or other vehicle. Applications may include software applications used by processor 50 or devices 52.

Communications manager system 36 may be used to communicate information between remote station 20 and physician station 24. According to one embodiment, communications manager system 36 may multiplex heterogeneous communication links in order to create a virtual path that may accommodate multimedia data in real time. According to the illustrated embodiment, communications manager system 36 includes a communications manager 60, database applications 62, and communication devices 64.

Communications manager 60 determines the capacity of communication links and makes a link selection in response to the determination. Communication manager 60 may also support dynamic bandwidth allocation across multiple communication devices 64 and support varying data rates based on the available communication links. For example, cellular digital packet data (CDPD) modems have a maximum throughput of 19.2 kbps, while Motorola data radios have a maximum data rate of 9.6 kbps. Communications manager 60 may adjust the system according to changes in communication link characteristics, data packet characteristics, or both. Communications manager 60 may also allow for handling link failure, tracking availability and throughput of communication devices 113, matching the bandwidth and channel requirements of applications 114 with available bandwidth, and instructing applications 114 to modify their bandwidth requirements in response to available bandwidth.

As an example operation, applications 44 may request various levels of reliability and different buffering times from communications manager 60. If maximum reliability is needed for data, the communications manager 60 buffers the data until the data can be sent. If partial reliability is needed, the data is buffered for a requested buffer time. If data need not be reliable, the data is not buffered.

Database applications 62 may be used to store data received at remote station 20 into a local database, and then transmit the data to physician station 24. Database applications 62 may include applications that allow remote station 20 to access information stored at an external database. Database applications 60 may comprise, for example, any suitable memory such as Random Access Memory (RAM), Read Only Memory (ROM), magnetic drives, disk drives, Compact Disk (CD) Drives, Digital Video Disk (DVD) drives, removable media storage, any other suitable data storage device, or a combination of any of the preceding.

Communication devices 64 may include, for example, a modem, a cellular telephone, a mobile handset, or any other device suitable for communicating data packets to and from system 10. Communication device 64 may support, for example, simple Internet Protocol (IP), mobile IP, or any other suitable communication protocol. Communication device 20 may utilize, for example, General Packet Radio Service (GPRS) technology or any other suitable mobile communication technology. A call from communication device 20 may comprise data packets communicating information such as data, video, multimedia, any other suitable type of information, or any combination of the preceding.

Power supply system 38 may be used to supply required current and voltage to the hardware components of remote station 20. Power supply system 38 may include a processor, devices, and applications. Devices may include, for example, an input-output device such as an analogue to digital converter for monitoring voltages. Applications may include software applications used by processor or devices to report power status to the user or to control power distribution.

Physician station 24 may allow information to be communicated between physician station 24 and remote station 20. According to the illustrated embodiment, a physician located at physician station 24 receives the information. Any suitable user, however, may receive the information. According to the illustrated embodiment, physician station 24 includes a processor 70, devices 72, servers/applications 74, and a communications manager system 76. Devices 72 may include medical or other equipment used to process medical information about the patient. Servers/applications 74 may include logic such as hardware or software that may be used by processor 70 or devices 72. Communications manager system 76 includes communications manager 80, a database 82, and communication devices 84, and may be substantially similar to communications manager system 36.

Third-party station 26 may include any suitable station with which remote station 20 or physician station 24 may communicate in order to send or receive medical information about the patient. As an example, third-party station 26 may include a database that stores the medical history of the patient or that stores patient records generated by remote station 20 or physician station 24. Medical history may provide information on, for example, pre-existing conditions, drug allergies, or other information.

Communication network 30 allows remote station 20, physician station 24, and third-party station 26 to communicate with each other. Communication network 30 may comprise a public switched telephone network (PSTN), a public or private data network, the Internet, a wireline or wireless network, a satellite link, a local, regional, or global communication network, an enterprise intranet, other suitable communication link, or any combination of the preceding.

Modifications, additions, or omissions may be made to system 10 without departing from the scope of the invention. The operations of the system may be performed by more or fewer modules. For example, the operations of communications manager 60 and database applications 62 may be performed by one module, or the operations of communications manager 60 may be performed by more than one module. Additionally, functions may be performed using any suitable logic comprising software, hardware, other logic, or any suitable combination of the preceding. As used in this document, “each” refers to each member of a set or each member of a subset of a set.

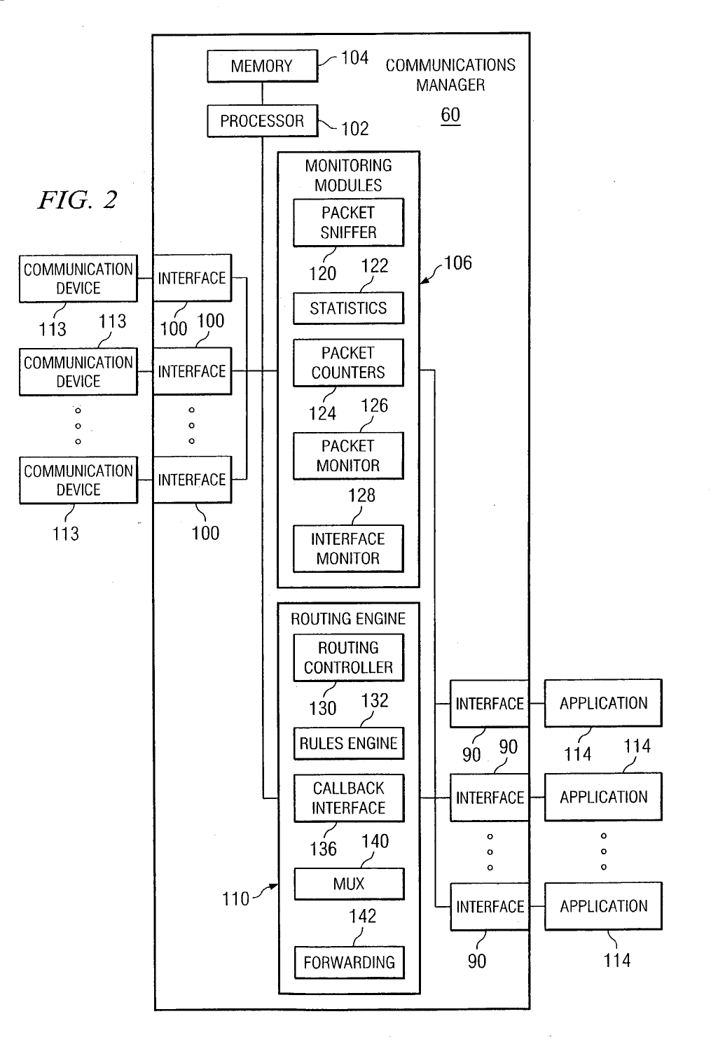

FIG. 2 is a block diagram illustrating one embodiment of communications manager 60 that may be used with system 10 of FIG. 1. Communications manager 60 determines the capacity of communication links and selects communication links in response to the determination, and may support varying data rates based on the available communication links. Communications manager 60 may adjust the communications links in response to changes in available bandwidth, required data, channel costs, delivery time, and priority. According to one embodiment, instructions received from remote station 20 or physician station 24 may override decisions by communications manager 60.

According to one embodiment, communications manager 60 manages multiple homogeneous or heterogeneous communication links to create virtual paths that provide logical communication paths between remote station 20 and physician station 24. A communication link may comprise an entity that defines a topological relationship between two nodes.

Examples of communication links include a cellular digital packet data (CDPD) communication link, a MOTOROLA DATATAC communication link, or a BREEZECOM communication link. A cellular digital packet data communication link may be provided using an advanced mobile phone system (AMPS). Cellular digital packet data modems, which may be serially connected to a computer via RS232, typically support data rates up to 19.2 kbps, and communicate via PPP/SLIP. DATATAC refers to a proprietary wireless packet data network that is commonly used by public safety departments. MOTOROLA modems, which may be serially connected to a computer via RS232, typically support data rates up to 9600 bps, and communicate via PPP/SLIP. The BREEZENET DS.11 technology may provide indoor client/server network configurations as well as long range point-to-multipoint links. BREEZENET DS.11 devices may use direct sequence, spread spectrum radio technology operating at a frequency of 2.4 to 2.4835 GHz and transmit data at a rate of 11 Mbps.

A communication link may be associated with a communication link characteristic that may operate to specify how data is communicated across the communication link. Examples of communication link characteristics include a specified portion of the communication spectrum, bandwidth, datalink protocol, network technology, actual throughput, operational cost of the communications link, or geographic availability of the device. Examples of datalink protocols may include the Ethernet, Point-to-Point (PPP), and AX.25 protocols. Examples of network technologies may include Wireless Fidelity (Wi-Fi), Integrated Services Digital Network (ISDN), General Packet Radio Service (GPRS), and packet radio technologies. Homogeneous communication links may have the same or substantially similar communication link characteristics, while heterogeneous communication links may be associated with one or more different communication link characteristics.

Homogeneous and heterogeneous communication links may be associated with homogeneous and heterogeneous, respectively, communication devices. A communication device may be associated with a communication device characteristic that specifies how the communication device communicates data packets. Examples of communication device characteristics may include a specified portion of the communication spectrum, a bandwidth, or other communication device characteristic such a form factor or physical interface type. Homogeneous communication devices may have the same or substantially similar characteristics, while heterogeneous communication devices may be associated with different characteristics.

Communications manager 60 may operate with any suitable combination of homogenous or heterogeneous communication links or communication devices. Communications manager 60 may use any suitable communication device supported by the host operating system, provided that communications manager 60 manager is able to enumerate the communication device. If a communication link has an associated communication device that is supported by the host operating system, communications manager 60 may integrate the capabilities of the communication link into its communications system.

According to one embodiment, communications manager 60 may route data streams at the packet level, and may support any suitable communication protocol such as the Internet Protocol (IP). Additionally, communications manager 60 may route data packets according to a set of rules. The rules may be used to determine the path and the manner according to which the data packets are to be routed. A callback procedure may be used to adjust the amount of bandwidth provided for the applications that request routing.

According to the illustrated embodiment, communications manager 60 includes one or more interfaces (IF) 90, a processor 102, a memory 104, monitoring modules 106, a routing engine 110, and one or more interfaces (IF) 100 coupled as shown in FIG. 2 that may be used to communicate data between one or more communication devices 113 and one or more applications 114.

Applications 114 send data to and receive data from communications manager 60 through interfaces 90. As an example, applications 114 may include software applications that may be used to generate and communicate medical information about a patient at remote station 20. According to one embodiment, middleware may be used between communications manager 60 and applications 114. The middleware may comprise a set of application programming interfaces that operate as an interface between communications manager 60 and applications 114. The middleware may be used to initiate connections, transfer messages, and close connections.

Processor 102 controls the operations of communications manager 60. Memory 104 stores data that may be used by processor, and may include Random Access Memory (RAM), Read Only Memory (ROM), magnetic drives, disk drives, Compact Disk (CD) Drives, Digital Video Disk (DVD) drives, removable media storage, any other suitable data storage device, or a combination of any of the preceding.

Monitoring modules 106 monitor the data packets routed by communications manager 60. According to the illustrated embodiment, monitoring modules 106 include a packet sniffer 120, a statistics module 122, a packet counter 124, a packet monitor 126, and an interface monitor 128. Packet sniffer 120 listens and captures data packets on the active interfaces 90. Packets may be captured using the LibPCAP library and sent to requesting modules for processing and routing. Statistics module 122 tracks current system statistics, for example, the number of data packets sent through each interface 100, interface 100 reliability, and latency through each interface 100.

Packet counter 124 tracks the number of data packets transmitted by communications manager 60, and may be used to determine if the data packets are lost. Packet monitor 126 may monitor data packet throughput and delay for each interface 90 and 100. Interface monitor 128 tracks the currently active interfaces 90 and 100, and may dynamically adjust interface lists and tables in order to reflect changes in the active interfaces 90 and 100.

Routing engine 110 provides routing support for applications 114. Routing engine 110 decides how data streams from applications 114 are to be transmitted to communication devices 113. According to one embodiment, routing engine 110 includes a routing controller 130, a rules engine 132, a callback interface 136, a multiplexer (MUX) 140, and a forwarding device 142.

Routing controller 130 uses rules engine 132 to determine how data streams are to be transmitted. Rules engine 132 maintains rules that are used to determine routing for data packets. A rule may comprise any suitable instruction embodied in logic such as hardware, software, or both that may be applied to cases in which certain characteristics are present. Examples of rules may include: if Wi-Fi communication is available, transmit data using only Wi-Fi communication; if the available bandwidth drops below a specific threshold, discontinue transmission of video packets; and always send patient telemetry over a dedicated communication device. According to one embodiment, certain rules may be overridden in response to a command from a user.

The routing decisions may be based upon rules applied to the current operational environment, which may be described by environmental characteristics. Examples of environmental characteristics may include individual interface bandwidth, total system bandwidth, interface latency and delay, interface device usage cost, communication device reliability, availability, compatibility, coverage area, quality of service, privacy, data composition, and data priority. Communication links with appropriate communication link characteristics may be selected to accommodate the operational environment.

Routing controller 130 may assign high capacity applications 114 that require high capacity to communicate data with high capacity communication devices 113 operable to communicate large amounts of data. Examples of high capacity communication technologies may include point-to-point, wireless LAN, Wi-Linx wireless LAN, LUCENT, mobile radio, and BREEZECOM technologies. Similarly, routing controller 130 may assign low capacity applications 114 that communicate smaller amounts of data to low capacity communication devices 113 that are operable to communicate smaller amounts of data. Examples of low capacity communication technologies may include cellular technologies, CDPD, METRICOM RICOCHET, ARDIS, PCS, CDMA, GSM, GPRS, TDMA, MOTOROLA DATATAC, QUALCOMM, and ERICSSON technologies.

Callback interface 136 controls the bandwidth requirements of each application 114. If the available aggregate bandwidth or the required bandwidth changes, routing controller 130 uses callback interface 136 to control the amount of data sent by applications 114. Routing controller 130 determines appropriate bandwidth allocation for each application 114, and sends the allocation to callback interface 136, which transmits instructions to applications 114. Callback interface 136 may, for example, instruct applications 114 to send more or less data.

Multiplexer 140 multiplexes data streams across homogeneous or heterogeneous interfaces 90 and 100 in response to feedback from rules engine 132. Forwarding module 142 forwards the data packets to the appropriate interface 90 and 100. Forwarding module 142 may fragment the data packets based upon the routing procedure. Interface 100 communicates packets to and from communication devices 113.

Communication devices 113 may include, for example, a modem, a cellular telephone, a mobile handset, or any other device suitable for communicating data packets to and from communications manager 60. Communication device 113 may support, for example, simple Internet Protocol (IP), mobile IP, or any other suitable communication protocol. Communication device 113 may utilize, for example, General Packet Radio Service (GPRS) technology or any other suitable mobile communication technology. A call from communication device 113 may comprise data packets communicating information such as data, video, multimedia, any other suitable type of information, or any combination of the preceding.

Modifications, additions, or omissions may be made to the system without departing from the scope of the invention. The operations of the system may be performed by more or fewer modules. For example, the operations of routing controller 130 and rules engine 132 may be performed by one module, or the operations of rules engine may be performed by more than one module. Additionally, functions may be performed using any suitable logic comprising software, hardware, other logic, or any suitable combination of the preceding.

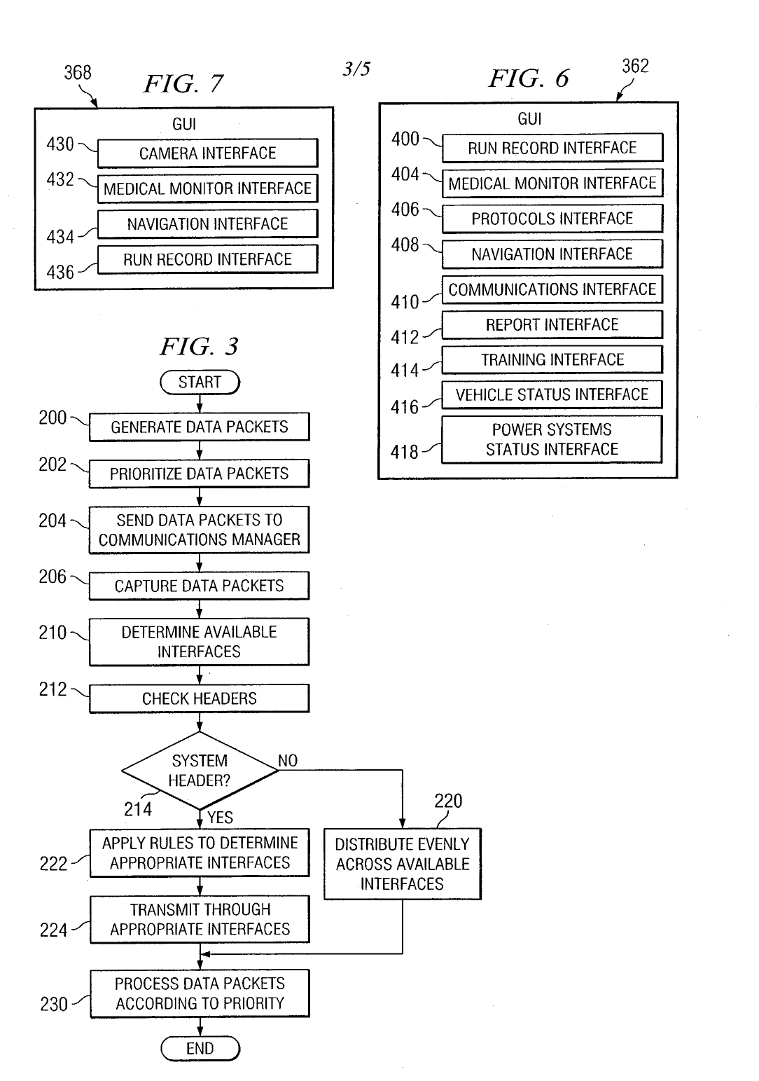

FIG. 3 is a flowchart illustrating one embodiment of a method for communicating data packets that may be used by communications manager 60 of FIG. 2. The method begins at step 200, where data packets are generated by applications 114. Each application 114 may generate data packets with a specific type of data having a specific requested reliability. The data packets may communicate medical information about a patient, and may be formatted according to any suitable communication protocol such as user datagram protocol (UDP).

The data packets are prioritized at step 202. Any suitable priority categorization may be used, for example, a priority categorization that includes three levels: a no priority level, a low priority level, and Ma high priority level. A no priority level may be associated with no packet delivery guarantee, a low priority level may be associated with a packet delivery guarantee as long as the communication connection is maintained, and a high priority level may be associated with a packet delivery guarantee regardless of whether the communication is maintained. The packet delivery guarantees may be provided for a data packet by sending a command to the recipient of the data packet to send an acknowledgment when the data packet is received.

A system specific header that denotes the type of data in a data packet, the reliability requirement of the data packet, the priority level of the data packet, other information, or any combination of the preceding may be appended to the data packet.

The data packets are sent to communications manager 60 at step 204. Packet sniffer 120 of communications manager 60 captures the data packets at step 206. Routing engine 110 determines the available interfaces 100 at step 210. The data packets are checked at step 212 to see whether they have the system specific header at step 214. If they do not have the system specific header, the method proceeds to step 220, where routing engine 110 distributes the data packets evenly across the available interfaces 100. If the data packets have the system-specific headers, the method proceeds to step 222. Routing controller 130 uses rules engine 132 to apply rules to determine the appropriate interfaces 100 through which to route the data packets. The data packets are transmitted through the appropriate interfaces 100 at step 224. The method then proceeds to step 230.

The data packets are further processed according to their priority at step 230. As an example, no priority data is sent to its destination and no acknowledgment is expected. For low and high priority data, a command is sent to the recipient along with the data packet. The command may be sent by retrieving a command index from the recipient's command queue and storing the command index in the packet header. The data packet is then added to the end of the recipient's command queue.

A send thread is created to monitor each recipient's command queue and repeatedly send the first available command in each queue until an acknowledgment of the command is received. When an acknowledgment is received, the command is removed from the head of the queue. For low priority packets, a timeout may be introduced. When the first command in the send queue has expired, the command is removed from the queue.

When the packet is received by the recipient, the recipient determines the priority level of the packet. The high priority packets may be automatically submitted to their destination for processing and an acknowledgment returned to the sender. For low priority packets, an acknowledgment may be sent for each command associated with the low priority packets. After processing the data packets, the method terminates.

Modifications, additions, or omissions may be made to the method without departing from the scope of the invention. Additionally, steps may be performed in any suitable order without departing from the scope of the invention.

FIG. 4 is a block diagram illustrating one embodiment of paramedic station 32 and physician station 24 that may be used with system 10 of FIG. 1. Devices 42 and servers/applications 44 of paramedic station 32 may operate with devices 72 and servers/applications 74 of physician station 24 to provide any suitable number of subsystems operable to provide functionality to system 10. As an example, subsystems may include an audio-video subsystem 300, a medical system 304, a power subsystem 305, an identification subsystem 306, a fleet subsystem 307, and a navigation subsystem 308. System 10, however, may include more, fewer, or other subsystems. The subsystems of system 10 may each have more, fewer, or other devices or servers/applications.

According to the illustrated embodiment, audio-video subsystem 300 includes devices and applications that may provide for the capture, compression, transmission, and display of audio or video data, for example, one or more cameras 310, an audio-video server 311, and camera control applications 312 of paramedic station 32, and telestration devices 314 and audio-video client 316 of physician station 24. Camera 310 may operate to capture audio and visual information describing a patient, and may comprise, for example, a camera that may be mounted at remote station 20 or on a tripod or that may be worn by a person.

Audio-video server 311 may be used to format, compress, noise suppress, and transmit audio or visual data such as video or voice data. The transmission may be voice-activated or manually triggered using an input device. Audio-video server 311 may be used to interface with audio-video hardware, which may involve accessing frame grabber hardware within paramedic station 32, interfacing with camera 310, compressing video frames, and managing multiple video and thumbnail streams from multiple devices 42. As an example, a region of a displayed image may be selected to focus video transmission bandwidth on that region to transmit that region at a higher resolution. Camera control 312 may be used to control camera 310 by, for example, zooming or tilting camera 310.

Audio-video client 315 may be used to provide audio visual information at physician station 24, and may allow a physician at physician station 24 to access camera control 312 of paramedic station 32 to remotely control camera 310. According to one embodiment, the same or similar images may be displayed at paramedic station 32 and physician station 24. Audio-video client 315 may allow the physician to set various settings such as the video frame rate, compression ratio, thumbnail refresh rate, and image size. Telestration application 314 may allow the physician to draw on the video image using, for example, a digital pen and screen, and display the drawings at remote station 20.

Medical subsystem 304 may include devices and applications that may be used to collect and communicate medical data about the patient, for example, medical equipment 320 and medical monitor server 321 of paramedic station 32 and medical equipment 322 and medical monitor client 324 of physician station 24. Medical equipment 320 and medical monitor server 321 may be used to provide information about the patient to physician station 24. Medical equipment 320 may include, for example, physiological telemetry equipment that captures physiological information such as electrocardiograms, SPO2, respiration, pulse, other vital sign, or any combination of the preceding. Medical equipment 320 may also include diagnostic equipment such as wet blood analysis chips, ultrasound, otolaryngoscope, other diagnostic equipment, or any combination of the proceeding.

Medical monitor server 321 may implement modules for accessing medical equipment 320 and provide an interface at remote station 20. Medical monitor server 321 may comprise any suitable server, for example, a vitals server for managing data communication between medical equipment comprising a vital signs monitor and other application modules. Medical monitor server 321 may allow the physician to remotely control the medical equipment 320 through medical monitor client 324.

Medical subsystem 304 may include any other suitable application related to the medical care of the patient. As an example, medical monitor applications 322 may include embedded medical protocols for paramedics and embedded training applications that emergency medical personnel may review during downtime.

Power subsystem 305 may include devices and applications that may be used to provide, monitor, and regulate a current and voltage supply. Power subsystem 305 may include, for example, a processor, a memory, monitoring modules 380 and interfaces, control modules, monitoring applications that start or stop hardware according to environmental conditions, and report applications at paramedic station 32 and at physician station 24.

Identification subsystem 306 may include devices and applications that may be used to identify a patient, a user of the system, medication, or other entity. Identification subsystem 306 may include, for example, an identification (ID) reader 330, a reader application 331, a medication scanner 332, and a scanner application 33 at paramedic station 32, along with an identification client 336 at physician station 24.

Identification reader 330 may be used to read identification of relevant users such as a patient, a paramedic, a system user, or an administrator. The identification reader 330 may comprise, for example, a magnetic card reader, and the identification may comprise, for example, an identification card such as a driver's license. Identification may be read when, for example, a patient is brought to remote station 20 or when a paramedic starts a new shift.

Reader applications 331 may include a card device class that initializes identification reader 330 and processes strings generated by identification reader 330. A parser class may be used to parse the strings by card type and field. Applications 114 may use a card listener interface to register for card events in the device class, such that when a card is read, the listeners are given the card string. As an example, the identification of the patient may be scanned in order to retrieve medical records associated with the patient.

Medication scanner 332 may be used to scan a medication identifier in order to record medication administered to the patient. Medication scanner 332 may comprise, for example, a barcode reader. When the medication is scanned, information about the medication may be retrieved and inserted into an appropriate field of a run record along with the dosage and time of administration. According to one embodiment, the time may be highlighted in order to indicate that the administration time needs to be verified by a user.

Scanner applications 333 may comprise a device driver class that initializes medication scanner 332, configures the serial port to which medication scanner 332 is coupled, and processes input data received when the medication is scanned. A listener class may provide an interface that defines the method called by the device driver class when a new medication identifier has been read.

Fleet subsystem 307 may include devices and applications that may be used to monitor the status of a vehicle (if any), control one or more of the operating parameters of the vehicle, or both. Fleet subsystem 307 may include, for example, monitoring modules 370, control modules, and applications 372 and 374 for reporting vehicle status to paramedic station 32 and to physician station 24.

Navigational subsystem 308 may include devices and applications that provide navigational functionality to generate or receive navigational information, for example, a global positioning system (GPS) receiver 340 and a map client 342 at paramedic station 32 and a map client 346 at physician station 24. Global positioning system receiver 340 may be used to capture the location of remote station 20 and transmit the location to physician station 24. Map client 342 and 346 may provide for map display and manipulation such as map zooming and map scrolling. Map client 342 and 346 may also track remote station 20 and display the location of remote station 20, the distance between remote station 20 and a stationary station such as physician station 24, and the estimated time of arrival of remote station 20 at the stationary station.

According to one embodiment, a device 42 located at remote station 20 may be controlled by a user located at physician station 24, or a device 72 located at physician station 24 may be controlled by a user located at remote station 20. As an example, a user at physician station 24 may enter commands that are received by a client at physician station 24. The client sends a control signal that includes the commands to a server located at remote station 20. The server controls device 42 in accordance with the control signal.

Paramedic station 32 may include any suitable input/output (I/O) devices 360 and a graphical user interface (GUI) 362, and physician station 24 may include any suitable input/output devices 364 and a graphical user interface 368. Input/output devices may comprise any device suitable to receive input or provide output. Examples of input/output devices may include a display, a monitor, a keyboard, a mouse, a speaker, a microphone, or any other device suitable for receiving or providing data.

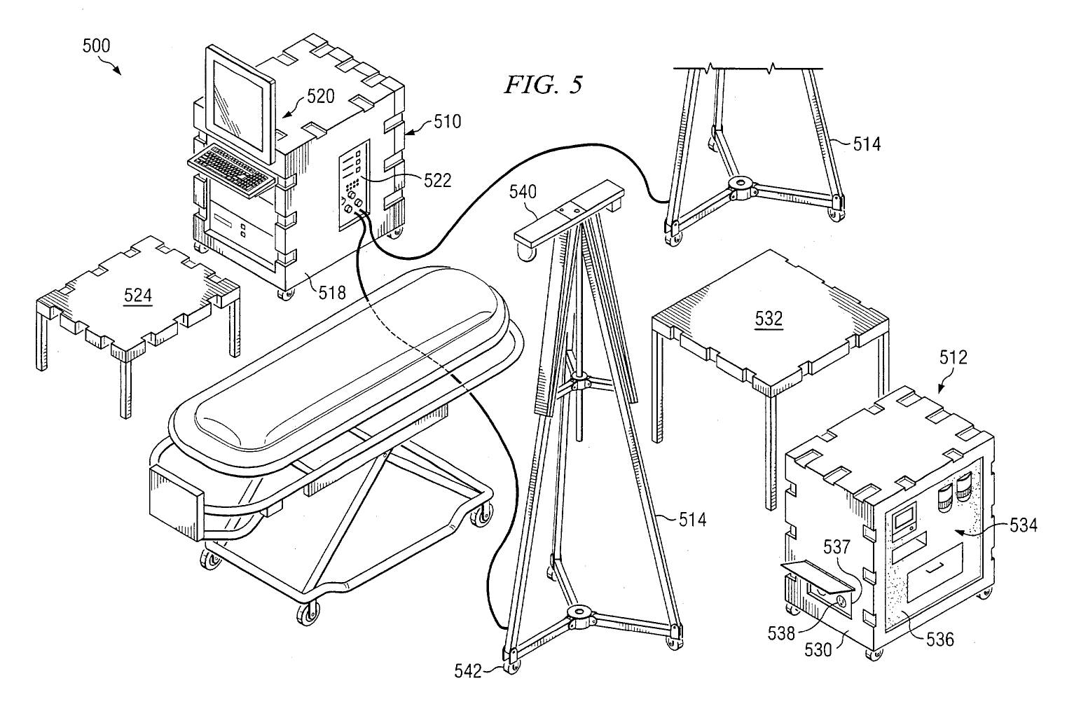

According to one embodiment, graphical user interfaces 362 and 368 may provide an integrated interface for one or more subsystems of FIG. 4. As an example, graphical user interface 362 may provide a user interface for any combination of audio-video subsystem 300, medical subsystem 304, identification system 306, and navigation subsystem 308. Examples of graphical user interface 362 and 368 are described in more detail with reference to FIGS. 5 and 6.

Modifications, additions, or omissions may be made to paramedic station 32 or physician station 24 without departing from the scope of the invention. The operations of paramedic station 32 or physician station 24 may be performed by more or fewer modules. For example, the operations of audio-video server 311 and camera control 312 may be performed by one module, or the operations of audio-video server 311 may be performed by more than one module. Additionally, functions may be performed using any suitable logic comprising software, hardware, other logic, or any suitable combination of the preceding.

FIG. 5 is a diagram illustrating one embodiment of graphical user interface 362 that may be used at paramedic station 32 of FIG. 4. According to the illustrated embodiment, graphical user interface 362 may include, for example, a run record interface 400, a medical monitor interface 404, a protocols interface 406, a navigation interface 408, a communications interface 410, a report interface 412, a training interface 414 a vehicle status interface 416, and a power system status interface 418. Graphical user interface 362, however, may include fewer, more, or other interfaces. The interfaces of graphical user interface 362 may be displayed in any suitable manner on any suitable display or combination of displays. As an example, run record interface 400 and medical monitor interface 404 may be displayed on the same or separate displays.

Graphical user interface 362 may display data generated using, for example, information received from the subsystems of FIG. 4 and from user input. The displayed data may include, for example, multi-media data generated by audio-video subsystem 300, information about the physiological condition of the patient generated by medical subsystem 304, the identification of the patient and of medications administered to the patient generated by identification subsystem 306, medical history and other medical information retrieved using the identification generated by identification subsystem 306, and location information generated by navigation subsystem 308.

Run record interface 400 may be used to generate a run record recording the description of the patient and the situation regarding the patent. As an example, the run record may include a time stamped log of medications administered to the patient. As another example, the run record may also include a narrative page in which the paramedic can enter narrative details of the incident scene and transport, treatment, and response of the patient. Run record, however, may include more, less, or other information.

Run record interface 400 may include one or more sections where information may be entered. As an example, a description of the incident section may include a portion where a vehicle such as a motorcycle, a sedan, or a semi may be selected and displayed. To indicate a patient's seat position in the vehicle, a seat of the vehicle figure may be selected and highlighted.

As another example, a medical condition section may include a list of vital signs with pull down menus from which an option describing the vital sign may be selected. The medical condition section may include a portion where a human figure that best matches the patient may be selected and displayed. Medical problems such as fracture, laceration, or swelling may be selected from one or more menus to create a numbered list of medical problems. A numbered label corresponding to a particular medical problem may be moved to a location of the human figure in order to indicate the presence of the medical problem at the location.

Medical monitor interface 404 may display a video of the patient as well as medical information about the patient. Protocols interface 406 may be used to access medical procedure protocols. Navigation interface 408 may be used to display navigational information such as the location of remote station 32. Communications interface 410 may display communication information and may allow the paramedic to adjust communication parameters. As an example, communications interface 410 may display maximum and actual throughput values, and may allow a paramedic to enable or disable a communication medium.

Report interface 412 may be used to display and modify run record report information. Report interface 412 may display a vital signs report, billing and insurance information, a release page, and a transport crew page. The vital signs report may include incident and vital signs information. The release page may include a release of responsibility and acknowledgment sections that a patient may use to acknowledge that medical care was or was not recommended and that the patient is accepting or denying care. The transport crew page may describe the patient transport method, the authorizing physician, and the destination of the patient.

Training interface 414 may allow a paramedic to receive computer-based training at paramedic station 32. The training may enable a paramedic to analyze case studies of emergency calls, test his or her knowledge of EMS protocols, or consult an online medical dictionary.

Vehicle status interface 416 may allow a user to monitor, control, or both various parameters of the vehicle operation. Parameters that may be monitored include battery voltage, engine temperature, alternator status, and fuel supply.

Power system status 418 may allow a user to monitor, control, or both various power supplies and power sources associated with paramedic station 32. Parameters that may be monitored include backup battery voltage, current, and temperature, and generator fuel supply, voltage, current, and temperature.

Modifications, additions, or omissions may be made to graphical user interface 362 without departing from the scope of the invention. The operations of graphical user interface 362 may be performed by more or fewer modules. For example, the operations of run record interface 400 and report interface 412 may be performed by one module, or the operations of medical monitor interface 404 may be performed by more than one module. Additionally, functions may be performed using any suitable logic comprising software, hardware, other logic, or any suitable combination of the preceding.

FIG. 6 is a diagram illustrating one embodiment of graphical user interface (GUI) 368 that may be used at physician station 24. According to the illustrated embodiment, graphical user interface 368 includes a camera interface 430, a medical monitor interface 432, a navigation interface 434, and a run record interface 436. Graphical user interface 368 may, however, include fewer, more, or other interfaces. The interfaces may be displayed in any suitable manner on any suitable configuration of displays. As an example, camera interface 430 and medical monitor 432 may be displayed on the same or separate displays.

Camera interface 430 displays images captured by cameras 310 at paramedic station 32, and may also allow a user at physician station 24 to remotely control cameras 310. As an example, a physician may use camera interface 430 to control camera 310 to change the images captured by camera 310. Medical monitor interface 432 displays medical information captured by medical equipment 320 at paramedic station 32, and may allow a user at physician station 24 to control medical equipment 320.

Navigation interface 434 displays the location of paramedic station 32, the distance between paramedic station 32 and a stationary location such as a hospital, the estimated time of arrival at the stationary location, other navigational information, or any combination of the preceding. Run record interface 436 displays the run record for the patient that may include information entered by the paramedics at paramedic station 32. The run record may include, for example, the physiological condition of the patient, the medical history of the patient, medications administered to the patient, other information, or any combination of the preceding.

Modifications, additions, or omissions may be made to graphical user interface 368 without departing from the scope of the invention. The operations of graphical user interface 368 may be performed by more or fewer modules. For example, the operations of camera interface 430 and medical monitor interface 432 may be performed by one module, or the operations of medical monitor interface 432 may be performed by more than one module. Additionally, functions may be performed using any suitable logic comprising software, hardware, other logic, or any suitable combination of the preceding.

FIG. 7 illustrates one embodiment of a deployable telemedicine system 500 for communicating medical information. System 500 may allow for medical information about a patient at remote station 20 to be communicated to a physician at physician station 24. According to the illustrated embodiment, system 500 includes a communications module 510, an equipment module 512, and one or more tripods 514 configured as shown in FIG. 5. Equipment module 512 may be used to gather medical information about a patient, and communications module 510 may be used to communicate the medical information to physician station 24. Although system 500 is illustrated as having two modules, system 500 may have any suitable number of modules, with the functionality of system 500 configured among the modules in any suitable manner.

Communications module 510 may include a housing 518, a computer 520, a power supply 522, and a lid 524. Communications module 510 may be carried by one or more humans, and have dimensions of less than 32 cubic feet, such as approximately less than nine cubic feet, and weigh less than 350 pounds, such as approximately less than 140 pounds. Housing 518 may comprise any material operable to provide ruggedized protection for communications module 510, for example, plastic, metal, or other sturdy material. Computer 520 may comprise a processor, applications, and input/output devices such as a display or keyboard. Power supply 522 may provide power for computer 520 and for external devices. Lid 524 may serve as a lid for housing 518. According to one embodiment, lid 524 may have one or more retractable vertical support structures such as legs and may operate as a seating device such as a stool.

According to the illustrated embodiment, equipment module 512 may include a housing 530, a lid 532, equipment 534, padding 536, a power distribution system 537, and a power supply 538. Equipment module 512 may be carried by one or more humans, and have dimensions of less than 32 cubic feet, such as approximately less than nine cubic feet, and weigh less than 350 pounds, such as approximately less than 140 pounds. Housing 530 may comprise any material suitable for providing ruggedized protection for equipment module 512 such as metal, plastic, or other material. Lid 532 operates as a lid for housing 530. According to one embodiment 532 may have retractable vertical support structures such as legs and operate as a table.

Equipment 534 may include medical diagnostic and monitoring devices, as well as audio-visual devices that may be used to communicate medical information about the patient. Padding 536 may comprise foam having recessed areas operable to receive separate pieces of equipment 534. Power supply 538 may provide power to equipment 534 and to external devices through power distribution system 537. Tripods 514 may include a head 540 and one or more rollers 542. Head 540 may be designed to receive a camera included in equipment 534. Rollers 542 may be used to position tripod 514.

Modifications, additions, or omissions may be made to the system without departing from the scope of the invention. The operations of the system may be performed by more or fewer modules. For example, the operations of communications module 510 and equipment module 512 may be performed by one module, or the operations of equipment module 512 may be performed by more than one module. Additionally, functions may be performed using any suitable logic comprising software, hardware, other logic, or any suitable combination of the preceding.

Certain embodiments of the invention may provide one or more technical advantages. A technical advantage of one embodiment may be that medical information may be communicated using heterogeneous communication links. A technical advantage of another embodiment may be that the medical information may be gathered using a portable system. A technical advantage of yet another embodiment may be that medical and multimedia data may be communicated in real time.

Although an embodiment of the invention and its advantages are described in detail, a person skilled in the art could make various alterations, additions, and omissions without departing from the spirit and scope of the present invention as defined by the appended claims.

What is claimed is:

1. A telemedicine system for communicating medical information, comprising:
one or more portable modules operable to receive medical information associated with a patient, the one or more portable modules comprising a plurality of devices comprising:
a medical device operable to collect the medical information comprising medical data associated with the patient;
an audio-video device operable to collect the medical information comprising multimedia data associated with the patient; and
a communication device coupled to the medical device and to the audio-video device and operable to communicate the medical information to a station in real time.

2. The telemedicine system of claim 1, the one or more portable modules further comprising:
an equipment module operable to collect the medical information, the equipment module comprising the medical device and the audio-video device; and
a communications module coupled to the equipment module and comprising the communication device.

3. The telemedicine system of claim 1, a portable module of the one or more portable modules comprising:
a padding material having an outer surface forming a recessed area operable to receive a device of the plurality of devices; and
a housing operable to house the padding material and the device of the plurality of devices.

4. The telemedicine system of claim 1, a portable module of the one or more portable modules comprising a housing having a lid, the lid comprising a plurality of retractable vertical supports operable to support the lid to form a table apparatus.

5. The telemedicine system of claim 1, a portable module of the one or more portable modules comprising a housing having a lid, the lid comprising a plurality of retractable vertical supports operable to support the lid to form a seating apparatus.

6. The telemedicine system of claim 1, a portable module of the one or more portable modules comprising:
a plurality of heterogeneous communication devices comprising a first communication device and a second communication device; and
a communications manager operable to multiplex a plurality of data packets associated with the first communication device and a plurality of data packets associated with the second communication device to communicate the medical information.

7. The telemedicine system of claim 1, a portable module of the one or more portable modules comprising an identification device operable to gather an identifier identifying the patient, the communications device operable to communicate the identifier identifying the patient to the station.

8. A method for communicating medical information, comprising:
receiving medical information associated with a patient at one or more portable modules, the one or more portable modules comprising a plurality of devices comprising a medical device, an audio-video device, and a communication device coupled to the medical device and to the audio-video device;
collecting the medical information comprising medical data associated with the patient using the medical device;
collecting the medical information comprising multimedia data associated with the patient using the audio-video device; and
communicating the medical information to a station in real time using the communication device.

9. The method of claim 8, the one or more portable modules further comprising:
an equipment module operable to collect the medical information, the equipment module comprising the medical device and the audio-video device; and
a communications module coupled to the equipment module and comprising the communication device.

10. The method of claim 8, wherein:
the communication device comprises a first communication device of a plurality of heterogeneous communication devices comprising the first communication device and a second communication device; and
communicating the medical information to a station in real time using the communication device comprises multiplexing a plurality of data packets associated with the first communication device and a plurality of data packets associated with the second communication device.

11. The method of claim 8, further comprising gathering an identifier identifying the patient using an identification device, the communications device operable to communicate the identifier identifying the patient to the station.

12. A telemedicine system for communicating medical information, comprising:
a portable equipment module operable to collect medical information associated with a patient, the equipment module comprising:
a medical device operable to collect the medical information comprising medical data associated with the patient;
an audio-video device operable to collect the medical information comprising multimedia data associated with the patient; and
a portable communications module coupled to the equipment module and comprising a communication device coupled to the medical device and to the audio-video device and operable to communicate the medical information to a station in real time.

13. The telemedicine system of claim 12, the equipment module further comprising:
a padding material having an outer surface forming a recessed area operable to receive the medical device; and
a housing operable to house the padding material and the medical device.

14. The telemedicine system of claim 12, the equipment module further comprising a housing having a lid, the lid comprising a plurality of retractable vertical supports operable to support the lid to form a table apparatus.

15. The telemedicine system of claim 12, the communication module further comprising a housing having a lid, the lid comprising a plurality of retractable vertical supports operable to support the lid to form a seating apparatus.

16. The telemedicine system of claim 12, the communication module further comprising:
a plurality of heterogeneous communication devices comprising a first communication device and a second communication device; and
a communications manager operable to multiplex a plurality of data packets associated with the first communication device and a plurality of data packets associated with the second communication device to communicate the medical information.

17. The telemedicine system of claim 12, the communication module further comprising an identification device operable to gather an identifier identifying the patient, the communications device operable to communicate the identifier identifying the patient to the station.

18. A system for communicating medical information, comprising:
means for receiving medical information associated with a patient at one or more portable modules, the one or more portable modules comprising a plurality of devices comprising a medical device, an audio-video device, and a communication device coupled to the medical device and to the audio-video device;
means for collecting the medical information comprising medical data associated with the patient using the medical device;
means for collecting the medical information comprising multimedia data associated with the patient using the audio-video device; and
means for communicating the medical information to a station in real time using the communication device.

19. A telemedicine system for communicating medical information, comprising:
one or more portable modules operable to receive medical information associated with a patient, the one or more portable modules comprising a plurality of devices comprising:
a medical device operable to collect the medical information comprising medical data associated with the patient;
an audio-video device operable to collect the medical information comprising multimedia data associated with the patient; and
a communication device coupled to the medical device and to the audio-video device and operable to communicate the medical information to a station in real time, the one or more portable modules further comprising:
an equipment module operable to collect the medical information, the equipment module comprising the medical device and the audio-video device; and
a communications module coupled to the equipment module and comprising the communication device, a portable module of the one or more portable modules comprising:
a padding material having an outer surface forming a recessed area operable to receive a device of the plurality of devices;
a housing operable to house the padding material and the device of the plurality of devices, the housing further comprising:
a first lid comprising a plurality of retractable vertical supports operable to support the first lid to form a table apparatus; and
a second lid comprising a plurality of retractable vertical supports operable to support the second lid to form a seating apparatus;
a plurality of heterogeneous communication devices comprising a first communication device and a second communication device;
a communications manager operable to multiplex a plurality of data packets associated with the first communication device and a plurality of data packets associated with the second communication device to communicate the medical information; and
an identification device operable to gather an identifier identifying the patient, the communications device operable to communicate the identifier identifying the patient to the station.
